(19) United States
(12) Patent Application Publication
(10) Pub. No.: US 2024/0341883 A1
(43) Pub. Date: Oct. 17, 2024

(54) BEDSIDE SETUP PROCESS FOR MOVABLE ARM CARTS IN SURGICAL ROBOTIC SYSTEM

(71) Applicant: Covidien LP, Mansfield, MA (US)

(72) Inventor: William J. Peine, Ashland, MA (US)

(21) Appl. No.: 18/682,177

(22) PCT Filed: Sep. 23, 2022
(86) PCT No.: PCT/IB2022/059048

§ 371 Date: Feb. 8, 2024

Related U.S. Application Data
(60) Provisional application No. 63/249,172, filed on Sep. 28, 2021.

(51) Int. Cl. A61B 34/37; A61B 34/00; A61B 34/30; A61B 90/00

(52) U.S. Cl. CPC A61B 34/37; A61B 34/25; A61B 90/361; A61B 2034/305; A61B 2560/0437

(57) ABSTRACT

A process for setting up a surgical robotic system having a plurality of mobile carts, each supporting a setup arm coupled to a robotic arm includes configuring the setup and robotic arms into desired configurations. In addition, an alignment module projecting an alignment pattern is rotated to a desired bedside angle. Thereafter, the mobile cart is moved to a surgical table until the alignment pattern is parallel to the surgical table with the setup and robotic arms locked into their configuration to couple to a corresponding access port.

### BACKGROUND

Surgical robotic systems may include a surgical console controlling one or more surgical robotic arms, each having a surgical instrument having an end effector (e.g., forceps or grasping instrument). In operation, the robotic arm is moved to a position over a patient and the surgical instrument is guided into a small incision via a surgical access port or a natural orifice of a patient to position the end effector at a work site within the patient's body.

Prior to commencing a surgical procedure, the surgical robotic systems are configured, which includes positioning the robotic arms at desired locations relative to the surgical table. This process is performed by the surgeon or a technician, which may be time consuming and error prone due to manual inputs. Proper positioning and setup around the surgical table is an important aspect of using surgical robotic systems with arms located on individual mobile carts.

### SUMMARY

This disclosure describes a setup process for a surgical robotic system for different type of surgical procedures. In particular, this system and method provide a quick and consistent way to align the arm carts around a surgical table to allow adequate access for the bedside staff and avoid collisions between the robotic arms.

Initially, the clinical team enters the type of procedure through a graphical user interface (GUI) displayed on one of the displays of the surgical robotic system and selects a desired bedside setup, which includes an orientation angle for each of the robotic arms and mobile carts. The surgical robotic system then asks the user to move the setup arms of the mobile carts and once adjustments are completed, the angles are locked in by the surgical robotic system. The user also sets each of the alignment modules to the correct angle as specified on the GUI screen. Each arm cart is then wheeled to the bed so that an alignment pattern line is parallel to the side of the surgical table. The mobile cart is then moved until the port latch of each of the robotic arm is at the correct access port disposed within the patient. This process is repeated for each of the robotic arms and mobile carts.

The advantage of this approach is that the mobile carts themselves are used as the alignment mechanism. The alignment pattern provides a consistent way to get the correct rotation of the mobile carts, and then the locked setup arm provides a way to confirm the translation of the mobile carts. The disclosed method provides more accurate and consistent setups of the robotic and setup arms and corresponding mobile carts than the current method where users approximate the location of the mobile carts based on GUI setup guides alone.

According to one embodiment of the present disclosure, a method for setting up a surgical robotic system is disclosed. The method includes configuring each robotic arm of a plurality of robotic arms from a storage state into a configured state, each robotic arm of the plurality of robotic arms is attached to a one mobile cart of a plurality of mobile carts. The method also includes rotating each alignment module of a plurality of alignment modules from a starting position to an aligned position, each of the alignment modules is coupled to one robotic arm of the plurality of robotic arms. The method further includes projecting an alignment pattern from each alignment module of the plurality of alignment modules and moving each mobile cart of the plurality of mobile carts along with a corresponding robotic arm to a corresponding position relative to a surgical table such that each of the alignment patterns is parallel to the surgical table.

Implementations of the above embodiment may include one or more of the following features. According to one aspect of the above embodiment, the method may also include displaying on a graphical user interface (GUI) a setup configuration for each mobile cart of the plurality of mobile carts. The setup configuration may include the configured state for each of the robotic arms and the aligned position for each alignment module of the plurality of alignment modules. The configured state may include an angle or a position for each joint of a plurality of joints of each robotic arm. The aligned position may include an angle for each alignment module of the plurality of alignment modules. The method may further include outputting a first notification on the GUI in response to each of the robotic arms being moved into the configured state. The method may also include outputting a second notification on the GUI in response to each alignment module of the plurality of alignment modules being rotated to the aligned position. The method may additionally include coupling each robotic arm of a plurality of robotic arms a corresponding access port.

According to another embodiment of the present disclosure, a method for setting up a surgical robotic system is disclosed. The method may include configuring a robotic arm attached to a mobile cart from a storage state into a configured state. The method also includes rotating an alignment module coupled to the robotic arm from a starting position to an aligned position. Additionally, the method includes projecting an alignment pattern from the alignment module and moving the mobile cart along with the robotic arm to a position relative to a surgical table such that the alignment pattern is parallel to the surgical table.

Implementations of the above embodiment may include one or more of the following features. According to one aspect of the above embodiment, the method may also include displaying on a graphical user interface (GUI) a setup configuration may include the configured state for the robotic arm and the aligned position for the alignment module. The configured state may include an angle or a position for each joint of a plurality of joints of the robotic arm. The aligned position may include an angle for alignment module. The method may also include outputting a first notification on the GUI in response to the robotic arm being moved into the configured state. The method may further include outputting a second notification on the GUI in response to each of the alignment module being rotated to the aligned position. The method may additionally include coupling the robotic arm to an access port.

According to a further embodiment of the present disclosure, a surgical robotic system is disclosed. The surgical robotic system includes a plurality of mobile carts, each of which includes a robotic arm and an alignment module configured to rotate and to project an alignment pattern. The system also includes a display configured to output a graphical user interface (GUI), which is configured to: display a setup configuration for each mobile cart of the plurality of mobile carts. The setup configuration may include a configured state for each of the robotic arms and an aligned position for each of the alignment modules. The GUI is also configured to output a first notification in response to each of the robotic arms being moved into the configured state and a second notification in response to each of the alignment modules being rotated to the aligned position.

Implementations of the above embodiment may include one or more of the following features. According to one aspect of the above embodiment, the configured state may include an angle or a position for each joint of a plurality of joints of the robotic arm. The aligned position may include an angle for alignment module. The GUI is further configured to output a first notification in response to the robotic arm being moved into the configured state. The GUI is also configured to output a second notification in response to each of the alignment module being rotated to the aligned position. The GUI is further configured to receive a user confirmation in response to each of the robotic arms being moved into the configured state and in response to each of the alignment modules being rotated to the aligned position.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of mobile carts of FIG. 1 positioned about a surgical table according to an embodiment of the present disclosure;

FIG. 6 is a flow chart of a method for configuring the surgical robotic system of FIG. 1 according to an embodiment of the present disclosure;

FIG. 7A shows a graphical user interface for configuring the surgical robotic system of FIG. 1 according to an embodiment of the present disclosure;

FIG. 7B shows a graphical user interface for configuring each of the surgical robotic arms of the surgical robotic system of FIG. 1 according to an embodiment of the present disclosure;

FIG. 8 is a plan schematic view of the mobile carts of FIG. 1 positioned in a storage state according to an embodiment of the present disclosure;

FIG. 9 is a plan schematic view of one of the mobile carts of FIG. 1 in a configured state with the remaining mobile carts of FIG. 1 positioned in a storage state according to an embodiment of the present disclosure;

FIG. 10 is a plan schematic view of two of the mobile carts of FIG. 1 in a configured state with the remaining mobile carts of FIG. 1 positioned in a storage state according to an embodiment of the present disclosure;

FIG. 11 is a plan schematic view of three of the mobile carts of FIG. 1 in a configured state with the remaining mobile cart of FIG. 1 positioned in a storage state according to an embodiment of the present disclosure;

FIG. 12 is a plan schematic view of the mobile carts of FIG. 1 positioned in a configured state according to an embodiment of the present disclosure;

FIG. 13 is a plan schematic view the mobile cart of FIG. 1 being moved into aligned position relative to the surgical table of FIG. 5 according to an embodiment of the present disclosure;

FIG. 14 is a plan schematic view of one of the mobile carts of FIG. 1 in the aligned position relative to the surgical table of FIG. 5 with the remaining mobile carts of FIG. 1 in a holding queue according to an embodiment of the present disclosure;

FIG. 15 is a plan schematic view of two of the mobile carts of FIG. 1 in the aligned position relative to the surgical table of FIG. 5 with the remaining mobile carts of FIG. 1 in a holding queue according to an embodiment of the present disclosure; and

FIG. 16 is a plan schematic view of three of the mobile carts of FIG. 1 in the aligned position relative to the surgical table of FIG. 5 with the remaining mobile cart of FIG. 1 in a holding queue according to an embodiment of the present disclosure.

### DETAILED DESCRIPTION

As will be described in detail below, the present disclosure is directed to a surgical robotic system, which includes a surgical console, a control tower, and one or more mobile carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

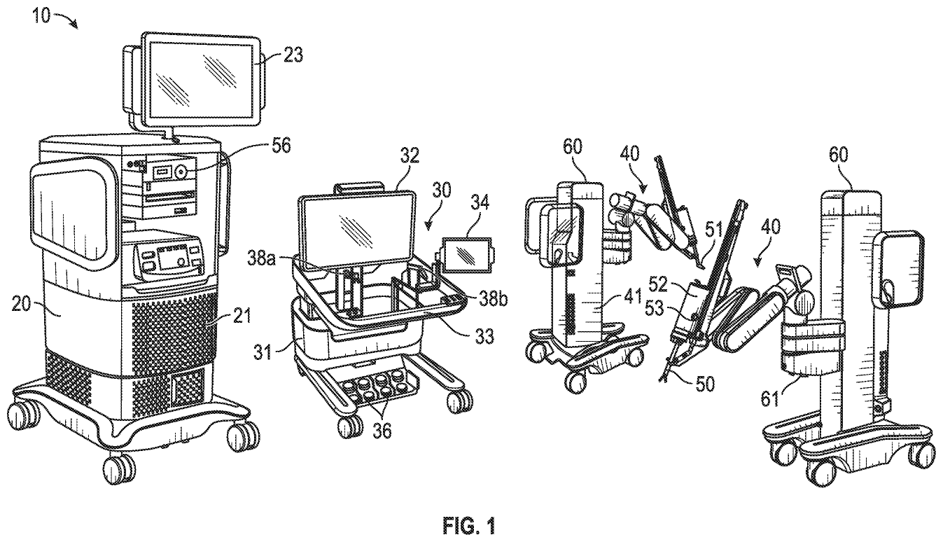

With reference to FIG. 1, a surgical robotic system 10 includes a control tower 20, which is connected to all of the components of the surgical robotic system 10 including a surgical console 30 and one or more movable carts 60. Each of the movable carts 60 includes a robotic arms 40 having a surgical instrument 50 removably coupled thereto. The robotic arms 40 is also coupled to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the present disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

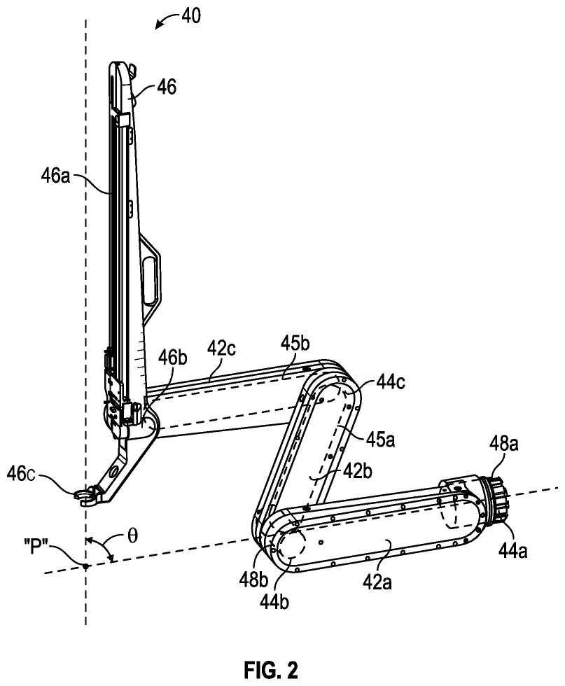

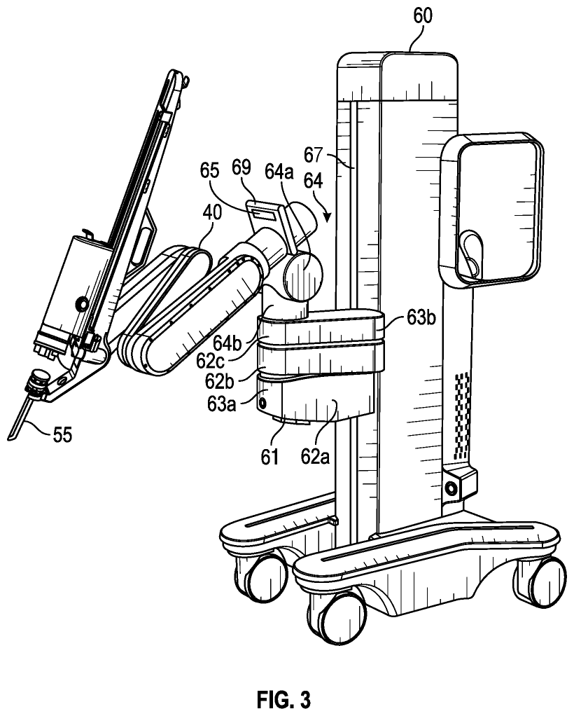

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46. The holder 46 also includes a port latch 46c for securing the port 55 to the holder 46 (FIG. 2).

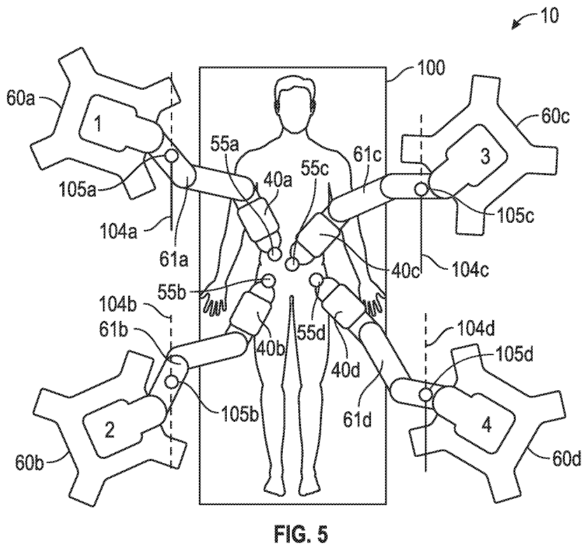

The robotic arm 40 also includes a plurality of manual override buttons 53 (FIGS. 1 and 5) disposed on the IDU 52 and the setup arm 61, which may be used in a manual mode. The user may press one or more of the buttons 53 to move the component associated with the button 53.

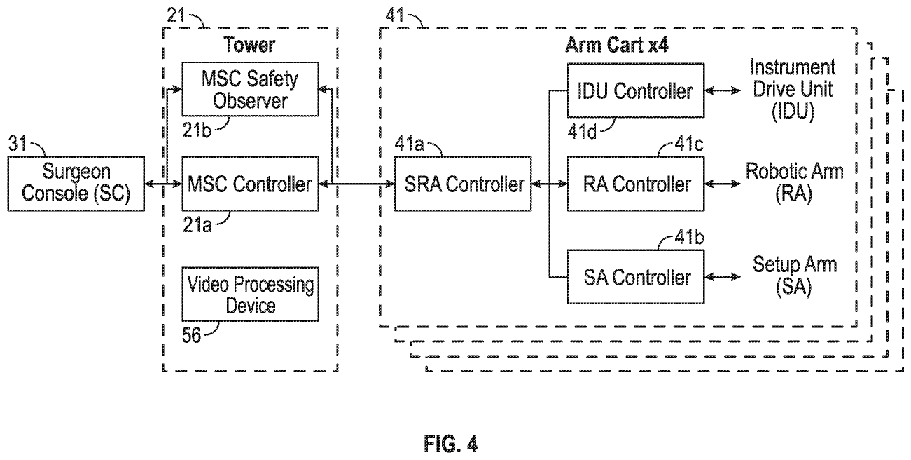

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 62 are passive joints (i.e., no actuators are present therein) allowing for manual adjustment thereof by a user. The joints 63a and 63b and the rotatable base 64 include brakes that are disengaged by the user to configure the setup arm 62. The setup arm controller 41b monitors slippage of each of joints 63a and 63b and the rotatable base 64 of the setup arm 62. when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (RPY) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

With reference to FIG. 5, the surgical robotic system 10 is setup around the surgical table 100. The system 10 includes mobile carts 60a-d, which may be numbered “1” through “4.” The mobile carts 60a-d may be positioned relative to the surgical table 100 and each other using any suitable registration system or method. During setup, each of the mobile carts 60a-d are positioned around the surgical table 100. Position and orientation of the mobile carts 60a-d depends on a plurality of factors, such as placement of a plurality of ports 55a-d, which in turn, depends on the procedure being performed. Once the port placement is determined, the ports 55a-d are inserted into the patient, and carts 60a-d are positioned and aligned relative to the surgical table 100. The setup arms 61a-d and robotic arms 40a-d of each of the mobile carts 60a-d are attached to the corresponding ports 55a-d and the instruments 50 as well as the endoscopic camera 51 are inserted into corresponding ports 55a-d.

Orientation of the mobile carts 60a-d may be based on individual alignment patterns 104a-d, which are projected onto a horizontal surface. The alignment patterns 104a-d may be projected onto any surface, such as a surgical table, a floor, patient, or any other surface. The surface may not be completely horizontal as long as the alignment patterns 104a-d projected onto the surface is visible and discernable by a clinician or a computing device. Accordingly, any non-vertical surface may be used.

The alignment patterns 104a-d are projected by alignment modules 105a-d disposed on respective mobile carts 60a-d. Each of the alignment modules 105a-d includes a projector for illuminating the alignment patterns 104a-d, which include an upper portion and a lower portion projected in different colors and/or patterns to allow for orientation of the alignment module 105a-d. Each of the alignment modules 105a-d is rotatable from 0-360° allowing for rotation of the respective alignment patterns 104a-d. Each of the alignment modules 105a-d also includes any suitable rotary position sensor for measuring the amount of rotation of the alignment modules 105a-d. The rotary position sensor may be any suitable encoder, potentiometer, rotary variable differential transformer, and the like.

Each of the alignment modules 105a-d is rotatable from a starting (i.e., zero) position in which each of the alignment patterns 104a-d is parallel with a front of the mobile carts 60a-d and the aligned position in which the alignment patterns 104a-d are parallel with the surgical table 100 and oriented in the same direction, i.e., first pattern is on the top. The alignment modules 105a-d are communicatively coupled to the computers 21, 31, and/or 41 transmitting the rotation data, namely, rotation from the starting position to the aligned position (e.g., from 0° to 45°) providing the system 10 with alignment information for each of the mobile carts 60a-d.

In embodiments other alignment methods may be used in lieu of patterns, such as sound indicators and/or flashing lights that beep and/or pulse with increasing frequency to indicate approximation to the desired position and continuous activation to indicate completion of the alignment.

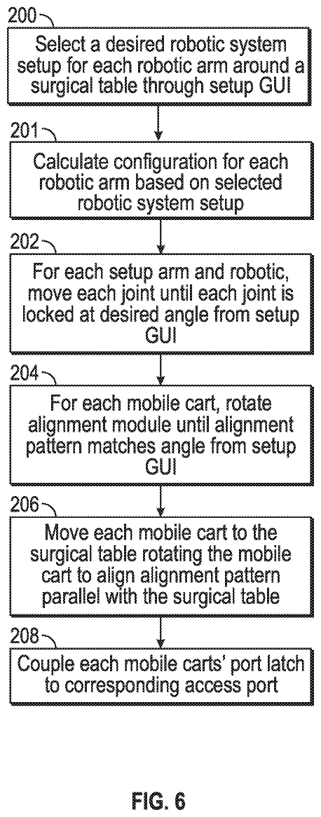

With reference to FIG. 6, a flow chart of a method for setting up the surgical robotic system 10 initially includes at step 200 selecting a desired setup configuration for the surgical robotic system 10 based on the procedure being performed. In particular, patient's habitus (i.e., body type, weight, height, gender, etc.) and the type of the procedure (e.g., cholecystectomy, appendectomy, etc.) may determine placement of the access ports 55a-d. Location of the access ports 55a-d may be determined using surgery planning software running on any computing device and then provided to the computer 21 of the control tower 20. The computer 21 may then determine the setup configuration for each of the mobile carts 60a-d and other components of the surgical robotic system 10.

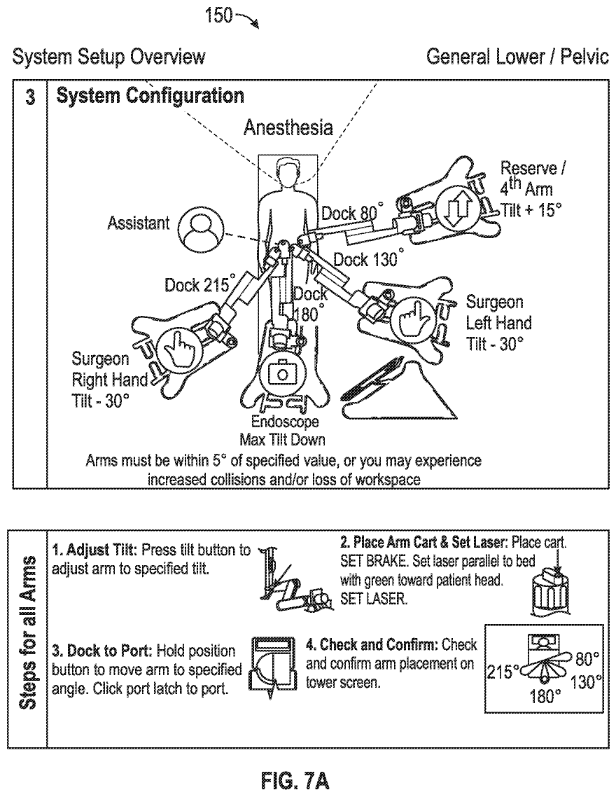

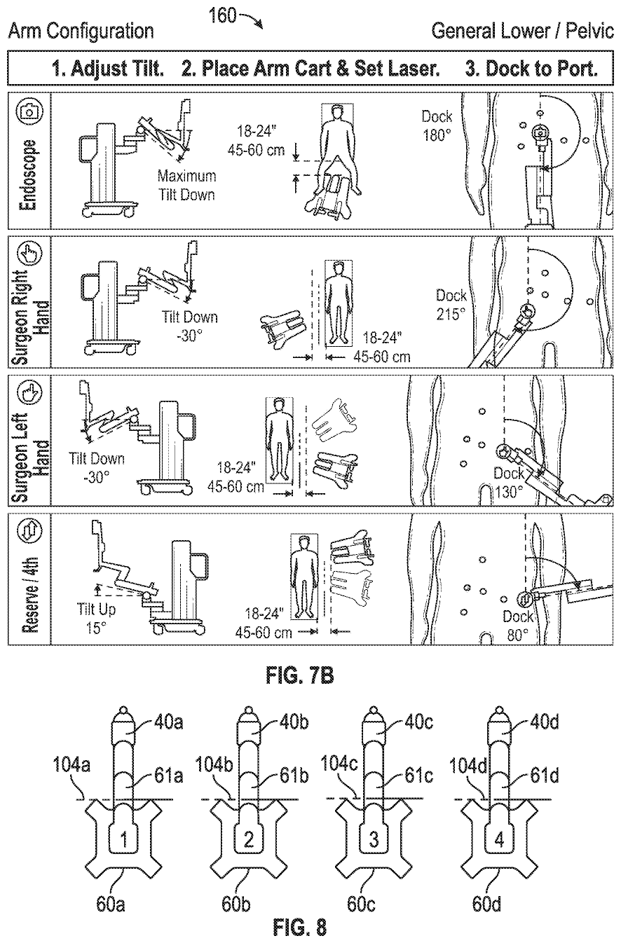

The setup may be aided using a GUI 150 as shown in FIG. 7A, which may be shown on the display 23 of the control tower 20. The GUI 150 shows location of the access ports 55a-d, orientation and position of mobile carts 60a-d and their respective setup arms 61a-d, as well as tilt angles for the robotic arms 40a-d. The GUI 150 also shows which of the robotic arms 40a-d are holding which instruments 50 and the endoscopic camera 51. More detailed instructions for each of the mobile carts 60a-d are shown in a GUI 160 of FIG. 7B.

At step 201, configuration for each of the setup arms 61a-d and the robotic arms 40a-d is calculated based on the selected system configuration at step 200. Configuration also includes rotation angles for each of the alignment modules 105a-d. The configuration calculations may be performed locally, e.g., at the controller 21a, or remotely, e.g., at a server, and provided to the controller 21a, which receives the configuration. The GUIs 150 and 160 are generated based on the received configuration and includes angles of each of the joints of the setup arms 61a-d and the robotic arms 40a-d as well as the alignment angle of the mobile carts 60a-d.

After the planning phase, including selecting the desired setup and calculating or receiving configuration for each of the robotic arms 40a-d, is complete, the operating room staff may proceed with configuring the surgical robotic system 10 according to the selected setup configuration. At step 202, each of the setup arms 61a-d and the robotic arms 40a-d are moved into desired configuration to achieve the orientation and pitch angles indicated by the selected setup configuration on the GUIs 150 and 160. This may be achieved manually, by placing each of the setup arms 61a-d and the robotic arms 40a-d into passive manual mode allowing for manipulation of each of joints 63a and 63b and 44a and 44b of the setup arm 61 and the robotic arm 40, respectively. The angles are provided on the GUIs 150 and 160 and the setup arms 61a-d and the robotic arms 40a-d are manipulated until the angles displayed angled are achieved. The GUIs 150 and/or 160 may output a notification that the desired configuration for the setup arms 61a-d and the robotic arms 40a-d has been achieved, which may be based on the provided angle feedback from the computers 41 of the mobile carts 60a-d. In additional embodiments, each of joints 63a and 63b and 44a and 44b of the setup arm 61 and the robotic arm 40, respectively, may be manipulated until a desired angle is achieved, at which point the main cart controller 41a locks the joints 63a, 63b, 44a, 44b. This may be done without relying on the GUI since the desired angles are used to lock the joints 63a, 63b, 44a, 44b.

In embodiments, each of joints 63a and 63b and the rotatable base 64 of the setup arm 62 may include actuators allowing for the setup arms 61a-d and the robotic arms 40a-d may be placed into selected setup configuration automatically, namely, by the controller 21a providing the setup configuration to the main cart controllers 41a to move the setup arms 61a-d and the robotic arms 40a-d into selected setup configuration. Thereafter, the setup arms 61a-d and the robotic arms 40a-d may be locked in to maintain their configurations. In further embodiments, this may be done automatically by the controller 21a, which would command the setup arms 61a-d the robotic arms 40a-d to move into the desired configuration.

Use of the alignment modules 105a-d may be optional. If in use, at step 204, each of the alignment modules 105a-d is activated to project their corresponding patterns 104a-d. Each of the alignment modules 105a-d is then rotated from the starting position, which may be indicated by physical markers, to the aligned position to achieve the desired orientation angle as indicated by the selected setup configuration. The alignment modules 105a-d are rotated until the angle displayed on the GUI 150 is achieved. The GUIs 150 and/or 160 may output a notification that the desired alignment has been reached. The alignment modules 105a-d may remain active, i.e., continuing to project the alignment patterns 104a-d or may go into a sleep mode to conserve energy until the setup arms 61a-d are moved to the surgical table 100 in step 206.

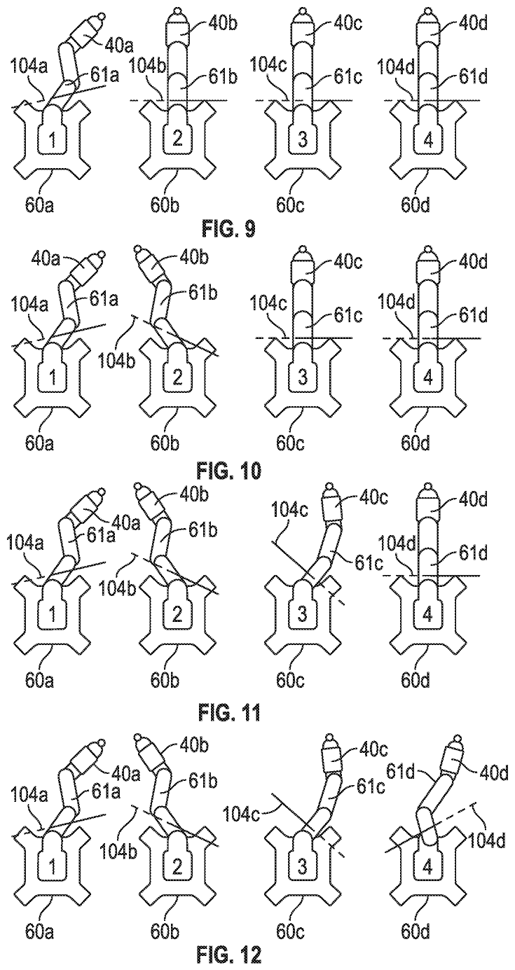

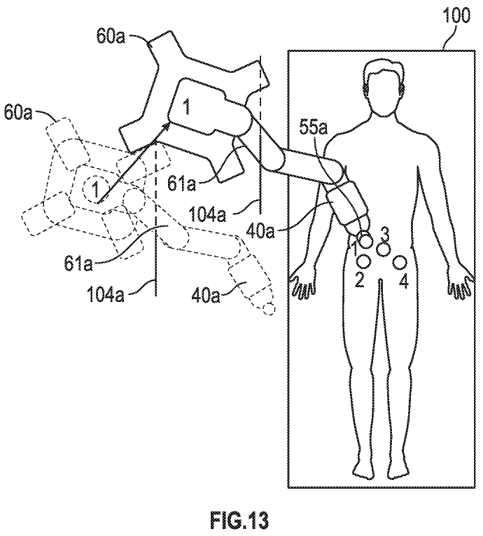

Configuration of each of the setup arms 61a-d and the robotic arms 40a-d and rotation of the alignment modules 105a-d is shown in FIGS. 8-12. As shown in FIG. 8, initially, each of setup arms 61a-d, the robotic arms 40a-d being disposed in straight (e.g., storage state) and the alignment modules 105a-d are projecting the alignment patterns 104a-d in the starting position, in which they are parallel to the front of the mobile carts 60a-d. FIGS. 9-12, show as each of the each of the setup arms 61a-d, the robotic arms 40a-d, and the alignment modules 105a-d are configured to achieve the desired angles and orientation.

At step 206, each of the mobile carts 60a-d is moved into their position as indicated on the GUI 150. The mobile carts 60a-d may be rotated and then moved into the position such that the projected alignment pattern 104a-d is parallel to the surgical table 100. This ensures that the setup arms 61a-d and the robotic arms 40a-d are disposed at the orientation angle indicated by the setup configuration on the GUI 150. Once the mobile carts 60a-d are in position, at step 208, the robotic arms 40a-d are coupled to the access ports 55a-d. Fine tuning the position of the setup arms 61a-d and/or the robotic arms 40a-d may be done to allow for the port latch 46c to attach and secure each of the access ports 55a-d. Thereafter, the mobile cart 60a-d may be secured in place by activating brakes (not shown). In addition, the operating room staff may confirm that each of the mobile carts 60a-d has been moved to the indicated position and each of the robotic arms 40a-d has been attached to the corresponding access port 55a-d. Confirmations may be entered through the GUIs 150 and/or 160. Various sensors and/or cameras may be used to confirm that the mobile carts 60a-d, the setup arms 61a-d, and the robotic arms 40a-d have been setup as indicated in setup configuration displayed in the GUIs 150 and 160. The confirmations are received by the controller 21a, which then sets that the mobile cart 60a-d and the robotic arms 40a-d are setup.

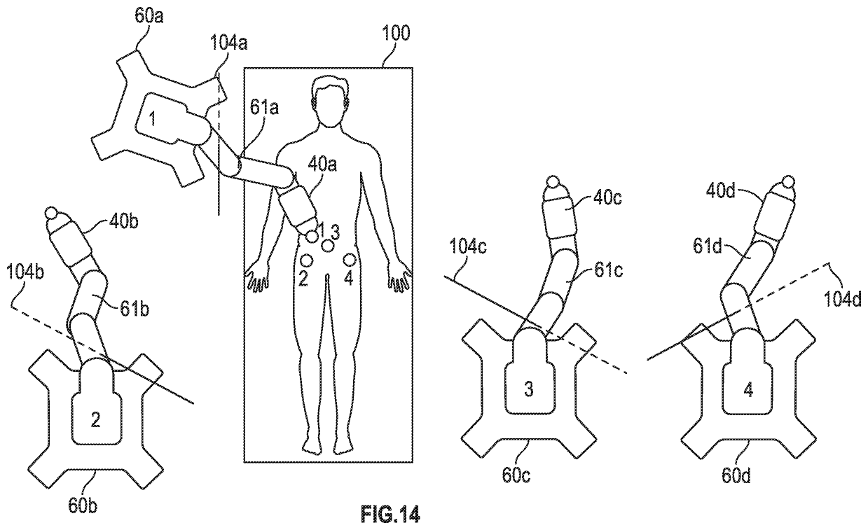

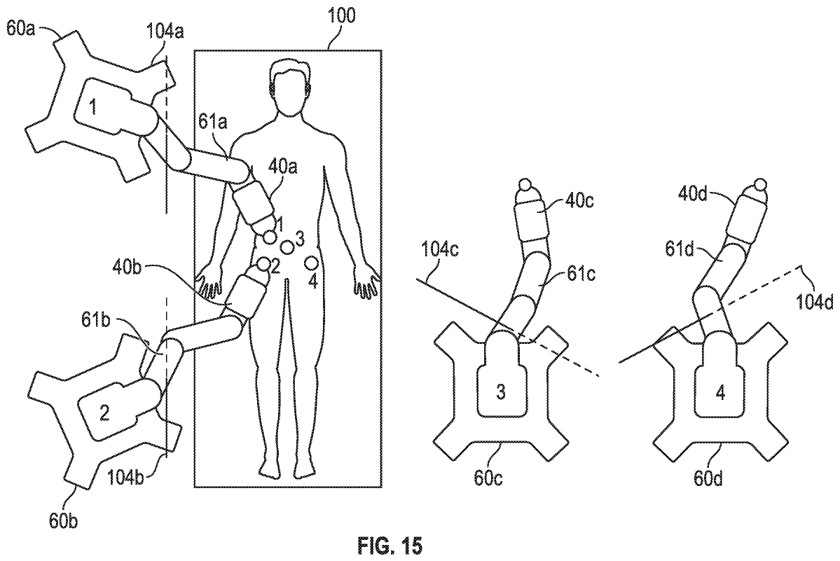

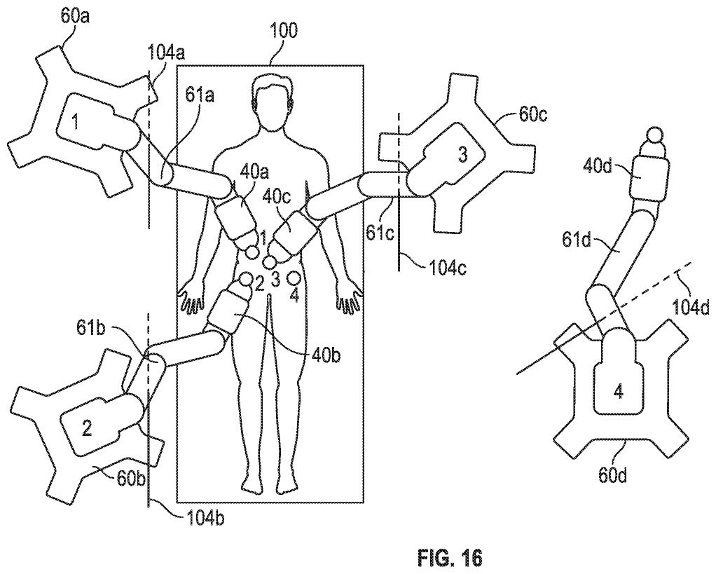

With reference to FIGS. 5 and 14-16, each of the mobile carts 60a-d is moved into position and latched to the access port 55a-d until the surgical robotic system 10 is configured as shown in FIG. 5, which matches the configuration indicated by selected setup configuration and as shown in the GUIs 150 and 160.

Prior to securing the robotic arms 40a-d to their respective access ports 55a-d, the position and configuration of the mobile carts 60a-d, the setup arms 61a-d, and robotic arms 40a-d may be fine-tuned and/or refined to achieve optimal placement around the surgical table 100 before docking to the access ports 55a-d. This may include localization of the mobile carts 60a-d relative to each other (position and orientation) and may be used to minimize collisions between robotic arms 40a-d to improve access during surgery. This refinement may be done manually or automatically if the joints are motorized. The refinement phase may be an optional phase entered in response to a prompt on the GUI 150, which the user may decline. If desired, the user may confirm and the robotic arms 40a-d and setup arms 61a-d may be unlocked to allow for additional movement. Once refinement movement is completed, the user may indicate as such on the GUI 150 to complete the process.

While the steps above are described according to a predetermined sequence, i.e., configuring the setup arms 61a-d and the robotic arms 40a-d prior to rotating the alignment modules 105a-d, this sequence is merely illustrative as the order of configuring each of the components of the surgical robotic system 10 is not material. Each or some of the components (e.g., mobile carts 60a-d) may be configured through each of the steps 202-208 before configuring the remaining components. Similarly, all of the components may be configured through each of the steps 202-208 together.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A method for setting up a surgical robotic system, the method comprising:
configuring each robotic arm of a plurality of robotic arms from a storage state into a configured state, each robotic arm of the plurality of robotic arms is attached to a one mobile cart of a plurality of mobile carts; and
receiving confirmation that each mobile cart of the plurality of mobile carts along with a corresponding robotic arm is located at a corresponding position relative to a surgical table such that each mobile cart of the plurality of mobile carts is aligned relative to the surgical table and each robotic arm of a plurality of robotic arms is coupled to a corresponding access port.

2. The method according to claim 1, wherein configuring includes providing instructions executable by a processor to each robotic arm of the plurality of robotic arms to move into the configured state.

3. The method according to claim 1, further comprising:
rotating each alignment module of a plurality of alignment modules from a starting position to an aligned position, each of the alignment modules is coupled to one robotic arm of the plurality of robotic arms;
projecting an alignment pattern from each alignment module of the plurality of alignment modules; and
moving each mobile cart of the plurality of mobile carts along with a corresponding robotic arm to the corresponding position relative to a surgical table such that each of the alignment patterns is parallel to the surgical table.

4. The method according to claim 3, further comprising:
displaying on a graphical user interface (GUI) a setup configuration for each mobile cart of the plurality of mobile carts, the setup configuration including the configured state for each of the robotic arms and the aligned position for each alignment module of the plurality of alignment modules.

5. The method according to claim 3, wherein the configured state includes an angle or a position for each joint of a plurality of joints of each robotic arm.

6. The method according to claim 3, wherein the aligned position includes an angle for each alignment module of the plurality of alignment modules.

7. The method according to claim 3, further comprising:
outputting a first notification on the GUI in response to each of the robotic arms being moved into the configured state; and
outputting a second notification on the GUI in response to each alignment module of the plurality of alignment modules being rotated to the aligned position.

8. A method for setting up a surgical robotic system, the method comprising:
configuring a robotic arm attached to a mobile cart from a storage state into a configured state;
rotating an alignment module coupled to the robotic arm from a starting position to an aligned position;
projecting an alignment pattern from the alignment module; and
moving the mobile cart along with the robotic arm to a position relative to a surgical table such that the alignment pattern is parallel to the surgical table.

9. The method according to claim 8, wherein configuring includes providing instructions executable by a processor to the robotic arm to move into the configured state.

10. The method according to claim 8, further comprising:
displaying on a graphical user interface (GUI) a setup configuration including the configured state for the robotic arm and the aligned position for the alignment module.

11. The method according to claim 9, wherein the configured state includes an angle or a position for each joint of a plurality of joints of the robotic arm and the aligned position includes an angle for alignment module.

12. The method according to claim 9, further comprising:
outputting a first notification on the GUI in response to the robotic arm being moved into the configured state.

13. The method according to claim 9, further comprising:
outputting a second notification on the GUI in response to each of the alignment module being rotated to the aligned position.

14. The method according to claim 8, further comprising:
coupling the robotic arm to an access port.

15. A surgical robotic system comprising:
a plurality of mobile carts, each of which includes a robotic arm and an alignment module configured to rotate and to project an alignment pattern; and
a display outputting a graphical user interface configured to:
display a setup configuration for each mobile cart of the plurality of mobile carts, the setup configuration including a configured state for each of the robotic arms and an aligned position for each of the alignment modules;
output a first notification in response to each of the robotic arms being moved into the configured state; and
output a second notification in response to each of the alignment modules being rotated to the aligned position.

16. The system according to claim 15, wherein the configured state includes an angle or a position for each joint of a plurality of joints of the robotic arm.

17. The system according to claim 15, wherein the aligned position includes an angle for alignment module.

18. The system according to claim 15, wherein the GUI is further configured to output a first notification in response to the robotic arm being moved into the configured state.

19. The system according to claim 15, wherein the GUI is further configured to output a second notification in response to each of the alignment module being rotated to the aligned position.

20. The system according to claim 15, wherein the GUI is further configured to receive a user confirmation in response to each of the robotic arms being moved into the configured state and in response to each of the alignment modules being rotated to the aligned position.
